(19) United States
(12) Patent Application Publication
(10) Pub. No.: US 2009/0069446 A1
(43) Pub. Date: Mar. 12, 2009

(54) DENDRITIC SALT THERAPEUTIC AGENT DELIVERY SYSTEM

(76) Inventor: Wayne Jeffrey PERRY, Schenectady, NY (US)

Correspondence Address: AMIN HALLIHAN, LLC, 444 NORTH ORLEANS STREET, SUITE 400, CHICAGO, IL, 60654, US

(21) Appl. No.: 12/205,308

(22) Filed: Sep. 5, 2008

Related U.S. Application Data
(60) Provisional application No. 60/970,266, filed on Sep. 6, 2007.

(51) Int. Cl. A61K 47/04

(52) U.S. Cl. 514/769

(57) ABSTRACT

A therapeutic agent delivery system includes a therapeutic agent, a dendritic salt used as a carrier to effectively deliver the therapeutic agent, and a salt base. The system can include the dendritic salt and the salt base in a ratio of about 1 part dendritic salt to about 8 parts salt base.

This application claims the benefit of earlier filed U.S. Patent Application Ser. No. 60/970,266 filed on 6 Sep. 2007.

### FIELD OF THE INVENTION

The present invention relates to a therapeutic agent delivery system containing dendritic salt used as a carrier to effectively deliver a therapeutic agent.

### BACKGROUND OF THE INVENTION

Dendritic salts are found naturally in salt flats and mines. They are predominantly found in the salt flats of Utah and Nevada. A dendritic salt is formed by the evaporation of brines that contain small amounts (approximately 5 ppm) of ferrocyanide ions. Dendritic salts may also be produced synthetically by combining ammonia, bluing, and then table salt.

In the past, dendritic salts have been used in salt baths which provide aromatherapy and other benefits to a human during exposure to such bath. However, stable non-caking delivery systems of high potency doses of essential oils and extracts, and delivery systems of essential oils which evenly deliver essential oils are unknown.

### SUMMARY OF THE INVENTION

Thus, one object of the present invention is a natural therapeutic agent delivery system.

Another object of the present invention is a therapeutic agent delivery system which effectively delivers a therapeutic agent by allowing the therapeutic agent to be fully soluble in the salt base carrier.

Still another object of the present invention is a therapeutic agent delivery system which will not degrade the therapeutic agent and will not interfere with the effectiveness of the therapeutic agent.

A further object of the present invention is a therapeutic agent delivery system wherein the therapeutic agent delivery system delivers high potency therapeutic agents in a stable environment.

A still further object of the present invention is a therapeutic agent delivery system wherein the dendritic salt crystals do not change and do not form a cake.

In accordance with the present invention, a therapeutic agent delivery system containing a dendritic salt is utilized to increase the permeability and the absorption of a therapeutic agent, while efficiently, quickly and effectively delivering the therapeutic agent to a human.

The therapeutic agent delivery system in accordance with the present invention, preferably, includes a dendritic salt.

In one embodiment of the present invention, the therapeutic agent delivery system includes dendritic salt, as a carrier for a therapeutic agent, and a salt base.

In another embodiment of the present invention, the therapeutic agent delivery system includes about 1 part dendritic salt, as a carrier for a therapeutic agent, and about 8 parts salt base.

Additionally, in the method of delivering a therapeutic agent using the therapeutic agent delivery system of the present invention, a dendritic salt is the carrier for the therapeutic agent. These and other embodiments of the present invention are more fully described in connection with the detailed description.

### DETAILED DESCRIPTION OF THE INVENTION

The present invention relates to a therapeutic agent delivery system containing a dendritic salt used as a carrier to quickly and effectively deliver a therapeutic agent.

Dendritic salts have low bulk density and high surface area and can be used to prevent clumping and/or to increase dissolution. Dendritic salts do not possess a cubic crystal shape as do other salts and, therefore, can trap additional ingredients, such as therapeutic agents, without additional moisture and caking. The six-sided cube shape of the dendritic salts allows the dendritic salts to absorb liquids without becoming wet. For example, essential oils are not fully soluble in water alone, therefore when delivered through dendritic salts the essential oils are delivered evenly.

Further, the dendritic salts take on the characteristics of therapeutic agents, such as essential oils and extracts, for easy delivery when added to water. Additionally, the dendritic salts allow a stable delivery for high potency doses of essential oils and extracts.

In one embodiment of the present invention, the dendritic salt is a naturally formed material. In another embodiment of the present invention, the dendritic salt is synthetically produced by combining ammonia, bluing, and then table salt. The ammonia-bluing solution provides the ferrocyanide ions which are necessary to create a dendritic salt. A dendritic salt possesses a low bulk density and high surface area and may be used to prevent clumping of cubic crystals of sodium chloride. Dendritic salt is essentially sodium chloride as are other salts, but the shape of the crystals trap essential oils without combining with additional moisture.

The present therapeutic agent delivery system is designed for intranasal, oral, or topical administration of a therapeutic agent. For intranasal administration, the therapeutic agent delivery system is designed to be added to a liquid, such as water, and used in a nasal rinse bottle, syringe, neti pot, and/or nasal rinse applicator. For oral administration, the therapeutic agent delivery system is designed to be added to a liquid, such as water, and used in throat gargles and mouth rinses and for topical administration, the therapeutic agent delivery system is designed to be used in body scrubs.

In another embodiment of the present invention, the therapeutic agent delivery system includes dendritic salt, as a carrier for a therapeutic agent, and a salt base. The therapeutic agent delivery system is not in liquid form but can be added to a liquid, such as water, for ease of delivery.

In yet another embodiment of the present invention, the therapeutic agent delivery system includes about 1 part dendritic salt, as a carrier for a therapeutic agent, and about 8 parts salt base. The about 1:8 ratio of dendritic salt to salt base allows the therapeutic agent to be delivered without moisture, caking, or uneven distribution.

The salt base of the present invention may be any salt of any grain size. The salt base of the present invention reduces the strength of the dendritic salt to effectively deliver the therapeutic agent without caustic reactions, or uncomfortable burning sensations from the therapeutic agent. The salt base may be a sea salt, a table salt, a pharmaceutical salt or any combination thereof. In a further embodiment of the present invention, the salt base is a sea salt to provide additional minerals when administered.

The therapeutic agent of the present invention may be in any amount or dosage. The therapeutic agent may be an herbal extract, an essential oil, a drug, or any combination thereof. The therapeutic agent may be administered through the present invention for treatment of a variety of ailments such as sinus relief, headache relief, allergy relief, facial pain associated with sinus inflammation and/or sore throat relief.

One embodiment of the present invention also relates to a method of delivering a therapeutic agent using the therapeutic agent delivery system described above and wherein a dendritic salt is a carrier for the therapeutic agent. The method includes the intranasal, oral or topical administration of a therapeutic agent with a dendritic salt as a carrier for the therapeutic agent.

The following are examples of formulations including a therapeutic agent and the therapeutic agent delivery system described above wherein the dendritic salt is a carrier for the therapeutic agent. It will be apparent to one of ordinary skill in the art that these are merely examples of formulations utilizing the therapeutic agent delivery system and many more such formulations are possible.

### EXAMPLE 1

One example of the present invention relates to a sinus relief composition for use with the therapeutic agent delivery system. The sinus relief composition provides prevention of sinus infections and relief of symptoms such as nasal allergies, headaches, and congestion. The sinus relief composition includes a dendritic salt and a salt base as the therapeutic drug delivery system; and eucalyptus oil, spearmint oil, black pepper oil, and lemon grass oil may comprise the therapeutic agent. The eucalyptus oil provides decongestion and the spearmint oil, blackpepper oil, and lemon grass oil provide cleansing properties for a long, lasting sensation. The salt base is a sea salt.

A sinus relief composition in accordance with the present invention for use with the therapeutic agent delivery system, includes:

one cup of dendritic salt crystals;

a therapeutic agent including: 2 mL of eucalyptus oil; 3 mL of spearmint oil; 1 mL of black pepper oil; and 1 mL of lemon grass oil; and

8 cups of sea salt base.

In another embodiment, the sinus relief composition may be mixed with water and administered intranasally through a nasal rinse bottle, syringe, neti pot or nasal rinse applicator. In one embodiment, about ½ to 1 teaspoon of the sinus relief composition may be mixed with about 4 to 8 ounces of water. In a further embodiment, the amount of the sinus relief composition may be adjusted depending upon the severity of the user's symptoms.

### EXAMPLE 2

One example of the present invention relates to a sore throat relief composition for use with the therapeutic agent delivery system. The sore throat relief composition provides sore throat relief. The sore throat relief composition includes dendritic salt and a salt base as the therapeutic drug delivery system; and slippery elm bark extract, wild cherry bark extract, echinacea extract, cayenne pepper extract, and spearmint oil may comprise the therapeutic agent. The slippery elm bark extract and wild cherry bark extract soothe sore throat pain and coughing; the echinacea extract boosts the immune system; the cayenne pepper extract relieves pain and inflammation; and the spearmint oil provides flavor. The salt base is a sea salt.

One example of the sore throat relief composition in accordance with the present invention for use with the therapeutic agent delivery system, includes:

one cup of dendritic salt crystals;

a therapeutic agent including: 3 mL of slippery elm bark extract; 3 mL wild cherry bark extract; 2 mL echinacea extract; 1 mL cayenne pepper extract; and 1 mL spearmint oil; and

8 cups of sea salt base.

In another embodiment, the sinus relief composition may be mixed with water and administered orally through a throat gargle or mouth rinse. In another embodiment, about 2 teaspoons of the sinus relief composition is used per about 1 cup of warm water. In a further embodiment, the amount of the sinus relief composition may be adjusted depending upon the severity of the user's symptoms.

In accordance with the present invention, when essential oils, herbal extracts or therapeutic agents are combined with any dendritic salt crystals in a variety of ways, they become soluble within the molecules without adversely changing the physical character of the dentritic salt crystal forms. This means that when any essential oil, herbal extract or pharmaceutical compound is combined with dentritic salt crystals up to the maximum soluble amount, the salt takes on the characteristics of the added ingredients including scent, taste and medicinal properties without compromising the stability of the salt crystals.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A therapeutic agent delivery system, comprising:
a dendritic salt, as a carrier for a therapeutic agent; and
a salt base.

2. The therapeutic agent delivery system in accordance with claim 1 further comprising:
about 1 part dendritic salt; and
about 8 parts salt base.
